(19) United States
(12) Patent Application Publication
(10) Pub. No.: US 2016/0220340 A1
(43) Pub. Date: Aug. 4, 2016

(54) DEVICE FOR OBTAINING A BITE REGISTRATION IMPRESSION

(71) Applicants: Roberto Jose Carrillo Gonzalez, Nuevo Leon (MX); Roberto Carrillo Fuentevilla, Nuevo Leon (MX)

(72) Inventors: Roberto Jose Carrillo Gonzalez, Nuevo Leon (MX); Roberto Carrillo Fuentevilla, Nuevo Leon (MX)

(21) Appl. No.: 14/611,629

(22) Filed: Feb. 2, 2015

(51) Int. Cl. A61C 19/05; A61F 5/56; A61C 9/00

(52) U.S. Cl. CPC A61C 19/05; A61C 9/0006; A61F 5/566

(57) ABSTRACT

A device for obtaining a bite registration impression which may be used to manufacture an intraoral appliance is disclosed. The device includes a support platform arranged to fit within a patient's mouth. The support platform includes upper and lower planar surfaces for retaining thereon an impression making material configured to be imprinted with an impression of the patient's teeth. A positioning block fits within a slot of the support platform and includes a plurality of upward facing bite grooves arranged for engaging dentition of the maxilla, and a plurality of downward facing bite grooves arranged for engaging dentition of the mandible. The positioning block is provided to position the mandible with respect to the maxilla, such as in a protruded position for creating an impression used in the manufacture a sleep apnea appliance.

### FIELD OF THE INVENTION

This invention relates generally to dental devices, and more particularly, but not by way of limitation, to a device for obtaining a bite registration impression which can be used in the manufacture of a variety of intraoral appliances, such as an appliance for treating sleep apnea.

### BACKGROUND OF THE INVENTION

Obstructive sleep apnea is a relatively common disorder which can produce morbidity and mortality. The disorder arises during sleep when the victim undergoes repeated cessation of breathing. This cessation results from an obstruction of the throat air passage (i.e., pharynx) due to severe narrowing or a collapse of the throat air passage. Without enough oxygen, the brain has to wake up the patient enough to start breathing again. The patient may or may not be aware that they have stopped breathing. A sleep mate will often hear the patient gasp or choke when they are able to breathe again. People with mild sleep apnea may stop breathing only a few times an hour. People with severe sleep apnea can stop breathing as many as 80 times an hour. Repeated cessation of breathing reduces blood oxygen and disturbs sleep. Reduction in blood oxygen can cause hypertension, heart attacks and strokes. Additionally, sleep disturbances can produce excessive daytime sleepiness, headache, depression, irritability and cognitive impairments.

Various oral appliances and methods have been proposed for the treatment of sleep disorders. Many such appliances place the mandible in a protruded position that maintains an open air way by preventing the lower jaw from falling back and collapsing the airway. This in turn will allow patients to inhale more air per breath leading to a better night's sleep. Such appliances treat sleep disorders such as sleep apnea without the need for surgery, a mask, or medication. Oral appliances are typically fabricated by dental laboratories and are dependent on a dentist providing impressions taken from the patient's upper and lower teeth as well as the bite registration for aligning the upper and lower teeth in the final appliance.

Often, the first step in the construction of such oral appliances is the determination of a bite registration that protrudes the mandible between a natural biting position and maximum protrusive position. Conventional practice is to advance the mandible between 60% to 80% of maximum voluntary protrusion. This step may be accomplished with a George Gauge™. The George Gauge™ is an intraoral device that allows the clinician to register the correct jaw position, e.g., protrusive jaw position, for mandibular repositioning appliances. The George Gauge™ measures the protrusion and retrusion of the mandible. A fast setting material is then used to obtain the bite registration based upon the desired protrusive jaw position.

The second step is determining the vertical dimension of occlusion that can be incorporated into the oral appliance. Although the George Gauge™ enables the clinician to determine the correct position of the mandible in the protrusive, retrusive directions, it only provides limited assistance in determining the vertical dimension, which is critical for maintaining an open airway. Consequently, the dental appliance created for the patient can sometimes need to be created several times before the appliance works correctly. And, in some cases, the patient becomes dissatisfied with the overall lack of good results and gives up. Thus, there is a need for a device for obtaining a bite registration impression that enables the dental clinician to position the dentition of the mandible in relation to the dentition of the maxilla in three dimensions.

### SUMMARY OF THE INVENTION

A device for obtaining a bite registration impression which may be used to manufacture an intraoral appliance is disclosed. The device includes a support platform arranged to fit within a patient's mouth. The support platform includes upper and lower planar surfaces for retaining thereon an impression making material configured to be imprinted with an impression of the patient's teeth. A positioning block fits within a slot of the support platform and includes a plurality of upward facing bite grooves arranged for engaging dentition of the maxilla, and a plurality of downward facing bite grooves arranged for engaging dentition of the mandible. The positioning block is provided to position the mandible with respect to the maxilla, such as in a protruded position for creating an impression used in the manufacture a sleep apnea appliance.

### DESCRIPTION OF THE DRAWINGS

FIG. 1 is an isometric view of the device for obtaining a bite registration impression which can be used in the manufacture of an appliance for treating sleep apnea;

FIG. 2 is an exploded isometric view of the device for obtaining a bite registration impression illustrating its two components including the support platform and the positioning block;

FIG. 2A is an elevational view of alternative embodiments of the positioning block of the present invention, each positioning block varying in height from the others;

FIG. 3 is an isometric view of the device for obtaining a bite registration impression with impression making material placed thereover;

FIG. 4 is a sectional view taken along line 4-4 of FIG. 3;

FIG. 5 is a side view of a mouth of a patient illustrating teeth of the maxilla and teeth of the mandible, with the mandible positioned in its natural biting position prior to insertion of the device of the present invention;

FIG. 6 is a side view, partially in section, of a mouth of a patient with the device of the present invention inserted therein to illustrate use of the device for creating a bite registration impression;

FIG. 7 is a side view, partially in section, of a mouth of a patient with the device of the present invention inserted therein to illustrate use of the device for creating a bite registration impression; and,

FIG. 8 is a top view of a resulting bite registration impression created by use of the device of the present invention.

### DETAILED DESCRIPTION OF THE PREFERRED EMBODIMENT

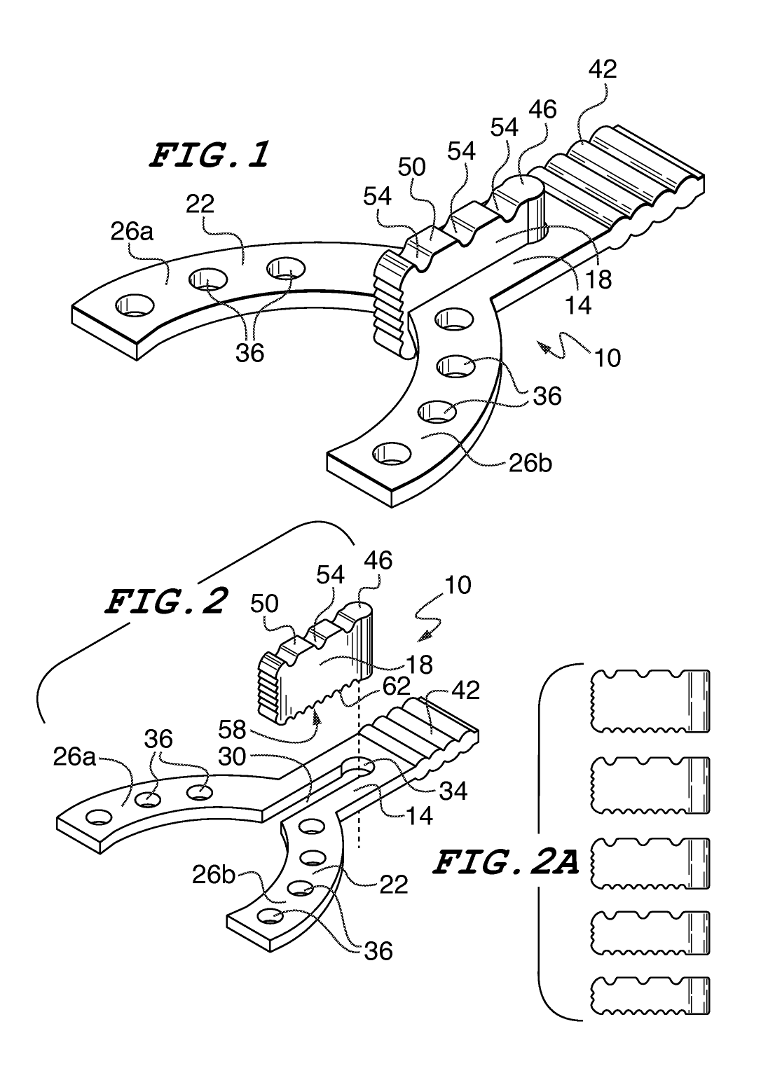

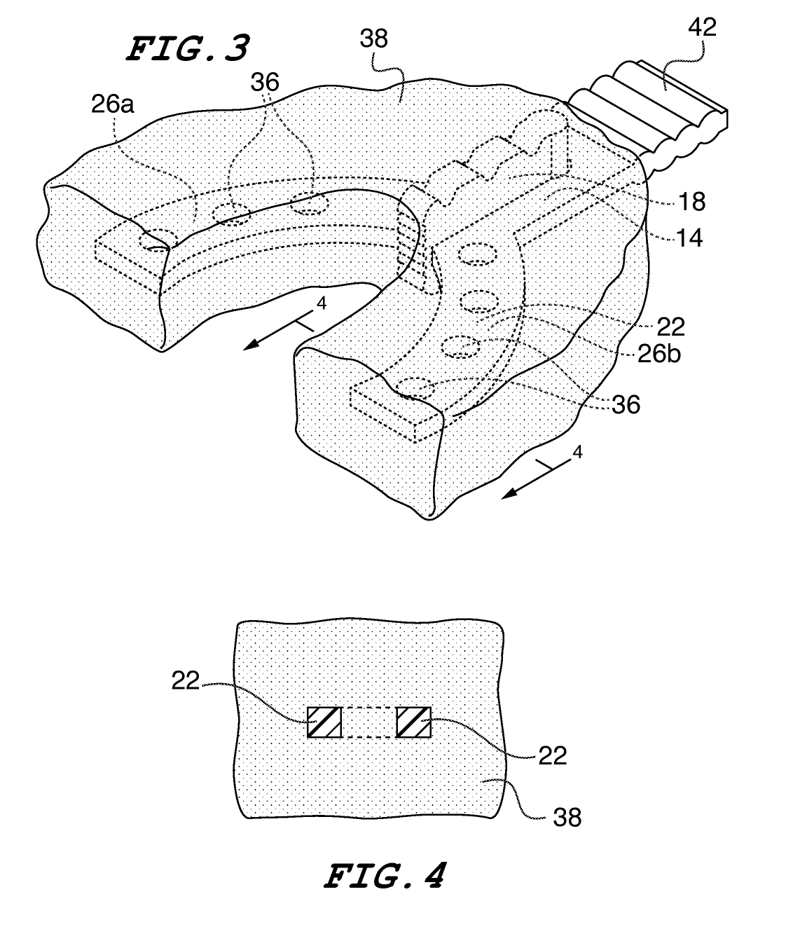

Referring now to FIGS. 1 and 2, wherein like numbers indicate like parts and components throughout the drawings, there is shown at 10 a preferred embodiment of the device for obtaining a bite registration impression of the present invention. The device 10 includes a support platform 14 and a positioning block 18. All parts may be made of plastic and/or metal. The support platform includes an internal end including a generally U-shaped mandibular arch 22 formed of opposed forks 26a and 26b. (In this description, the term “internal” refers to inside the mouth or toward the inside of the mouth, and “external” refers to outside the mouth or toward the outside of the mouth.) As best shown in FIG. 2, the forks 26a and 26b are spaced apart by a predetermined distance to define an elongated slot 30 located therebetween, the slot extending distally to a keyhole opening indicated at 34. The U-shaped mandibular arch 22 includes upper and lower planar surfaces on which a plurality of retention holes 36 are located to help secure an impression making material 38 onto the support platform 14. At the external end of the support platform 14, a handle 42 is provided which extends from the U-shaped mandibular arch 22. The handle 42 may be integral with the U-shaped mandibular arch 22 or connected thereto. As shown in FIGS. 1 and 2, the handle 42 may include a scalloped surface to facilitate placement of the device 10 into the mouth of a patient and removal of the device 10 therefrom. The impression making material 38 is used in the casting of dental impressions (FIG. 8) for use in creating an appliance for treating sleep apnea, and other uses. As should be apparent to someone skilled in the art, any thermoplastic, alginate or vinylpolysiloxine-based dental impression putty would be suitable for this purpose.

Referring again to FIGS. 1 and 2, the positioning block 18 may be a solid structure and may include a rounded end 46 arranged to fit through the keyhole opening 34 located at the distal end of the elongated slot 30 of the support platform 14. The positioning block 18 is arranged to be inserted into and frictionally engage with the elongated slot 30. The positioning block 18 is formed of an upper registration surface 50 which includes a plurality of bite grooves 54 and a lower registration surface 58, also including a plurality of bite grooves 62. Referring now to FIG. 2A, the positioning block 18 could be provided as a set of positioning blocks 18 of varying height. That is, the distance between the upper registration surface 50 and the lower registration surface 58 of the positioning block 18 could be varied for each positioning block 18 in the set for the purpose of controlling the vertical dimension of occlusion to be incorporated into the oral appliance.

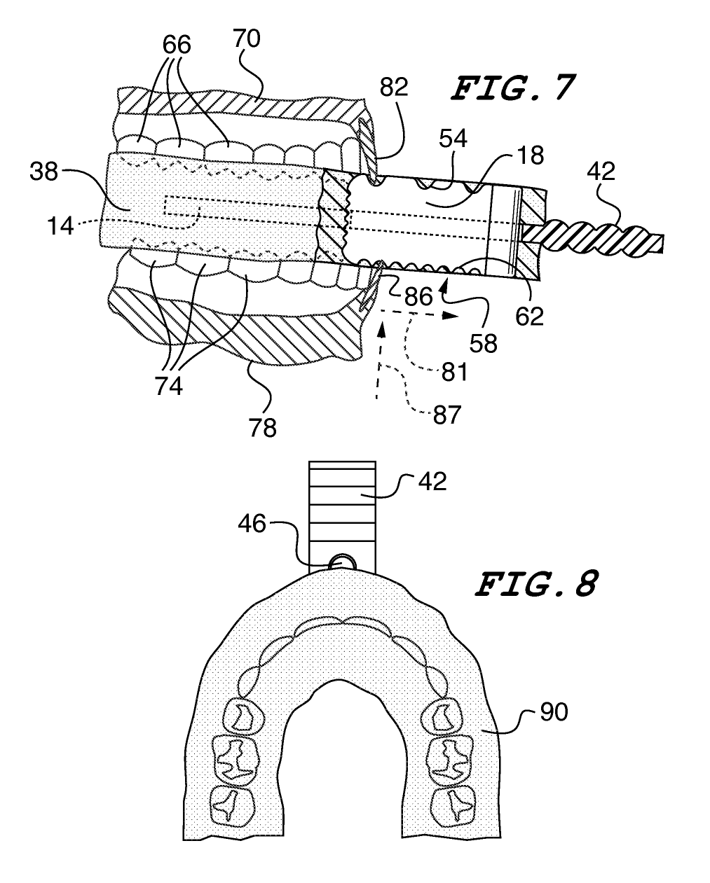

Referring now to FIG. 7, the bite grooves 54 are arranged for receiving one or more incisors 66 of the maxilla, while the bite grooves 62 are arranged for receiving one or more incisors of the mandible of a patient. The number of bite grooves 54, 62 located on the upper and lower registration surfaces 50, 58 in the figures is merely exemplary, and it is contemplated that a greater or fewer number of bite grooves 54, 62 could be utilized without departing from the scope of the invention. However, by providing numerous bite grooves in the upper and lower registration surfaces, a dental practitioner can precisely select an appropriate bite groove for placement of the incisors of the maxilla and mandible to obtain a suitable protruded position for obtaining a registration impression for use in creating an appliance for treating sleep apnea.

The series of steps which comprise the method of the present invention for obtaining a bite registration impression will now be discussed. First, as shown in FIGS. 1 and 2, a positioning block 18 of suitable height is selected and frictionally fitted into the slot 30 of the support platform with the rounded end 46 of the positioning block 18 inserted through the key hole 34 of the elongated slot 30 to obtain a proper positioning of the positioning block 18 within the slot 30. Referring now to FIG. 3, the next step in the procedure is to cover all surfaces of the internal portion of the support platform 14 as well as all surfaces of the positioning block 18 positioned therein with a supply of pliable impression making material 38. The impression making material can be formed of any suitable material commonly utilized for this purpose in the dental profession, e.g., an alginate or polyvinyl silaxane (PVS).

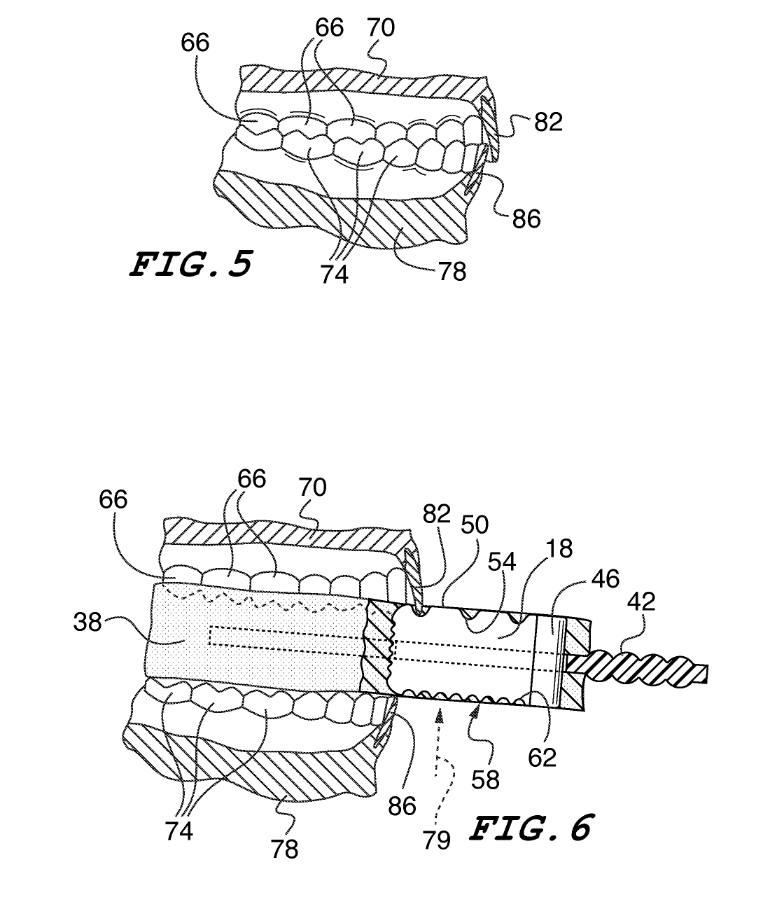

FIG. 5 illustrates the patient's mouth prior to insertion of the device 10 of the present invention, the patient's mouth including dentition 66 of the maxilla 70 and dentition 74 of the mandible 78, the mandible 78 being shown in the natural biting position. Referring now to FIG. 6, the dental clinician then grips the handle 42 of the support platform 14 and, while holding the handle 42, asks the patient to open his mouth and inserts the device 10 into the patient's mouth between the maxilla 70 and the mandible 78.

The device 10 is then seated in the patient's mouth and pressed against the dentition 66 of the maxilla 70 in the direction of arrow 79 so that the impression making material 38 contacts and conforms to the dentition 66 of the patient's maxilla 70 only (i.e. the incisors, cuspids, and molars) while the handle 42 extends externally to the patient's mouth. As best shown in FIG. 6, one or more incisors 82 of the maxilla 70 contact the impression making material 38 and extends into one of the plurality of bite grooves 54 located on the upper registration surface 50. Still referring to FIG. 6, the mandible 78 is shown therein in its natural biting position. As this stage, the dentition 74 of the mandible 78 has not yet contacted the impression making material 38 of the device 10.

A recommended amount of mandible protrusion is then determined, or has previously been determined. As discussed above, the conventional practice for determining a recommended amount of mandible protrusion is to advance the mandible between 60% to 80% of maximum voluntary protrusion. Once that recommended amount has been determined, the dental clinician advances the mandible 78 to that position of protrusion (as indicated by arrow 81) and the patient is then instructed to bite firmly on the impression making material 38 and into the bite groove 62 of the positioning block 18. Referring now to FIG. 7, as the patient bites (as indicated by arrow 87), the incisors 86 of patient's mandible 78 make contact with the impression making material 38 and extend into one of the plurality of bite grooves 62 of the lower registration surface 58 corresponding with the recommended amount of mandible protrusion. Likewise, the dentition 74 of the mandible 78 contacts the impression making material 38 which conforms to the dentition 74 of the patient's mandible 78, thus creating a bite registration impression 90 (FIG. 8) which can be used to manufacture an appliance for treating sleep apnea or any other device for mandibular positioning.

The device 10 should be left in the patient's mouth long enough to allow the impression making material 38 to set. After the impression making material 38 has set sufficiently to hold its own shape, utilizing the handle 42, the dentist may remove the device 10 from the mouth of the patient.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. It should be understood that the illustrated embodiments are exemplary only, and should not be taken as limiting the scope of the invention.

I claim:

1. A device for obtaining a bite registration impression for use in the manufacture of an appliance for treating sleep apnea, the device comprising:
a. a support platform arranged for fitting into the mouth of a patient and having upper and lower planar surfaces for retaining thereon an impression making material configured to be imprinted with and maintain an impression of the patient's teeth, said impression platform being generally U-shaped, and formed of two leg portions extending proximally from the incisors to the molars and a central portion located between said leg portions, said central portion including a slot having an open proximal end and a closed distal end; and,
b. a positioning block arranged to be received within the slot of said support platform, and including a plurality of upward facing bite grooves arranged for engaging incisors of the maxilla and a plurality of downward facing bite grooves arranged for engaging incisors of the mandible to position the mandible in relation to the maxilla in three dimensions so that an impression of the patient's teeth may be obtained with the mandible so positioned upon the patient biting down on the impression making material.

2. The device of claim 1, wherein said support platform includes a plurality of apertures located around its generally U-shaped configuration for enhancing the connection of the impression making material to the impression platform.

3. The device of claim 2, wherein said plurality of apertures further comprises a plurality of circular through-holes spaced evenly along the U-shaped configuration of said impression platform.

4. The device of claim 1, wherein said slot includes a keyhole opening at its closed distal end.

5. The device of claim 1, wherein said impression making material comprises at least one copolymer of polyethylene and polyvinyl acetate.

6. The device of claim 1, wherein said impression making material is a pliable material.

7. The device of claim 1, wherein said impression making material is a rapid setting material.

8. The device of claim 1, wherein said impression making material is removable from said support platform after an impression of the surfaces of the teeth of the maxilla and mandible has been obtained.

9. The device of claim 1, wherein the plurality of bite grooves included in the upward facing surface of said positioning block is less in number than the plurality of bite grooves located in the downward facing surface of said positioning block.

10. The device of claim 1, wherein said support platform and said positioning block are made of a material selected from the group consisting of plastics, composite, ceramics, and mixtures thereof.

11. The device of claim 1, additionally comprising a handle extending from said support platform and protruding out of the patient's mouth during use of said device, said handle being sized and shaped to permit gripping by an individual for inserting and removing said device from the patient's mouth for obtaining a bite registration impression.

12. The device of claim 1, wherein said positioning block further comprises a set of positioning blocks, each positioning block in the set having a different predetermined height.

13. A method for obtaining a bite registration impression for use in the manufacture of an appliance for treating sleep apnea, comprising the steps of:
a. providing an impression platform arranged for fitting into the mouth of a patient, the impression platform having upper and lower planar surfaces for retaining an impression making material, and being generally U-shaped and having two end portions extending proximal to the molars and a central portion including a slot having an open proximal end and a closed distal end;
b. providing a positioning block including an upward facing surface including a plurality of bite grooves arranged for engaging dentition of the maxilla and a downward facing surface including a plurality of bite grooves arranged for engaging dentition of the mandible;
c. properly orienting and sliding the positioning block within the slot of the impression platform;
d. placing an uncured impression making material onto the upper and lower surfaces of the impression platform;
e. moving the patient's mandible between protruded and retruded positions to obtain a recommended protrusion of the mandible with respect to the maxilla in the sagittal direction for obtaining the bite registration;
f. inserting the dental bite registration mold into the patient's mouth;
g. having the patient bite firmly onto the positioning block with the mandible in the recommended position for obtaining the bite registration such that dentition of the patient's maxilla engage bite grooves of the upward facing surface of the positioning block and dentition of the patient's mandible engage bite grooves of the downward facing surface of the positioning block;
h. conforming the impression making material to the patient's maxillary and mandibular dentition;
i. allowing the impression making material to cure with the patient biting firmly on the positioning block to obtain castings of the patient's teeth; and,
j. removing the dental bite registration mold from the patient's mouth.
